(19) United States
(12) Patent Application Publication
(10) Pub. No.: US 2024/0024710 A1
(43) Pub. Date: Jan. 25, 2024

(54) NOSE PLUG WITH ANTI-MICROBIAL ACTIVITY

(71) Applicant: NoseOption AB, Stockholm (SE)

(72) Inventor: Adrian LILJEFORS, Stockholm (SE)

(21) Appl. No.: 18/258,484

(22) PCT Filed: Dec. 16, 2021
(86) PCT No.: PCT/EP2021/086171

§ 371 Date: Jun. 20, 2023

(30) Foreign Application Priority Data
Dec. 22, 2020 (SE) 2051529-2

(51) Int. Cl. A62B 23/06

(52) U.S. Cl. CPC A62B 23/06

(57) ABSTRACT

A nose plug includes at least one flexible member comprising an anti-microbial substance, and one rigid, elongated support member. The flexible member is arranged to surround the support member, and is adapted to be placed within a nostril to accurately fill the opening of the nostril. The flexible member is provided with means for enabling air flowing in a tortuous structure facilitating inactivation of infectious agents by the anti-microbial substance.

### TECHNICAL FIELD

The disclosure relates to a nose plug comprising at least one flexible member and one rigid, elongated support member and having anti-microbial activity.

### BACKGROUND

People working in the health care area are often exposed to droplets or aerosols comprising infectious agents such as bacteria and viruses. During the worldwide spread of the virus Sars-Cov-2 concerns about transmission of this virus, and others, in all types of physical meetings also outside healthcare facilities have been raised.

This has led to widespread use of face masks in everyday life to prevent spread through droplets and aerosols, also in countries where use of face masks in public spaces has previously not been common. Face masks work by physically stopping the infectious agent from entering the nasopharyngeal cavity of the wearer, and also stopping infectious agents being exhaled by the user from spreading to people in the vicinity, or being transferred to the environment.

It is therefore fundamental that the face mask is worn in a way that ensures that essentially all air being inhaled or exhaled, and potentially comprising infectious agents, passes through the face mask so that the infectious agents are immobilized. Most face masks do however not inactivate the infectious agent. A face mask worn in a contaminated environment may thus comprise a substantial amount of viable infectious agent and must be treated as a biohazard. Proper use of face masks thus require education of users in how to put on, wear, take off and dispose of the face mask in a safe manner that ensures effective reduction of transmission of infectious agents. The use of face masks is also perceived by some potential users as anti-social due to the need to cover the user's face, hampering recognition by others and communication through facial expressions.

Other measures for reducing the risk of being infected by, or risk infecting others with, an infectious agent include physical isolation and social distancing. Such measures may however pose mental health risks to people if necessary for an extended period of time.

A less conspicuous manner of removing various types of particles in air inhaled by a user is using a filter mounted in a holder, which is placed in or over the nostrils, and where the inhaled air is directed through a mechanical filter. Such filters are known for various uses, such as filtering out smog particles or allergens, but are not normally very effective against smaller particles such as viral or bacterial aerosols. Furthermore, such filters are conceived to be hard to breathe comfortably through by some users.

Thus, it would be desirable to have access to some means for solving, among others, the above mentioned problems.

### SUMMARY

There is a need for a product that can prevent spread of microbial, such as bacterial, viral and fungal, infections. The product should be easy to use, easy to breathe through, easy to handle and cover a minimal area of a user's face, i.e. it should be a small and convenient product.

The object of the present disclosure is to address the problems outlined above. These objects, and others, are achieved by the apparatus according to the appended independent claim.

The present inventors have surprisingly found that a nose plug configured substantially as described in WO2011/162677 (incorporated by reference herein), further comprising an anti-microbial substance, i.e. a substance capable of inactivating infectious agents, has an unexpected high effect of minimizing the amount of microbial particles in the air inhaled and also exhaled by the user. Such a substance is thus preferably capable of inactivating and/or preventing viruses, bacteria or fungi from reaching a host or being spread further in the environment. The targeted infectious agents may be a virus, bacteria or fungi.

The anti-microbial substance may be incorporated in a material in contact with an air stream passing through the nose plug, or may be coated on a surface in contact with an air stream passing through the nose plug. The anti-microbial substance is preferably permanently immobilized in the material or on the surface. That is, the substance is preferably not released under normal usage and storage conditions.

According to a first aspect, the nose plug comprises at least one flexible member comprising an anti-microbial substance and one rigid, elongated support member. The flexible member is arranged to surround the support member, and the flexible member is adapted to be placed within a nostril to accurately fill the opening of the nostril. However, despite the plug filling the nostril, it is provided with means for guiding the flow of air entering the nostril in a serpentine or spiral like path by means of a plurality of plates or a band. Thus, when placed in the nostril of a user, the user experiences generally unhindered air flow when both inhaling and exhaling. In fact, in some cases, the user will experience enhanced ease of breathing, due to the slight expansion of the nostril when the nose plug is arranged in a nostril.

Air entering or exiting the nose is forced a longer distance than without the nose plug arranged in the nose, and is brought into contact with the anti-microbial substance comprised in the flexible member along this distance. The nose plug according to the present disclosure may thus be useful in reducing the microbial load of inhaled and/or exhaled air, and in methods for preventing, or reducing the risk or severity of, microbial infections.

In one aspect, the anti-microbial substance is permanently immobilized in the material of the flexible member or on the surface of the flexible member.

In one aspect, the anti-microbial substance is releasable from the material of the flexible member or from the surface of the flexible member.

In one aspect of a nose plug, a set of flexible members, arranged to surround the support member, comprises a plurality of parallel plates arranged along the support member and where each plate extends in a direction perpendicular to the extension of the support member. There is provided for communication between the spaces formed by adjacent plates, such that the air is forced to pass between the plates whereby, the air is forced to flow an extended distance. The communication is preferably provided in that each plate has one recess at the periphery, and the recesses are arranged such that no recess overlaps a recess in a previous or subsequent plate. The flow of air is thus, being forced to pass through the openings created by the recesses and makes it easy to breathe through the nose plug.

In one aspect the plates have a rounded shape, preferably elliptical. The plates may also have different sized peripheries. This is a preferred shape of the plates for the nose plug to properly adapt within a nostril.

In a preferred aspect of a nose plug a first set of recesses are arranged on every second plate on a position at the periphery at one end of the transverse diameter and a second set of recesses are arranged on every plate there between at the periphery at the other end of the transverse diameter. The distance of the air flowing into the nose will be as long as possible, which is advantageous in order to efficiently bring any infectious agents in the inhaled or exhaled air in contact with a large surface of the flexible member(s) where they may be inactivated.

In one aspect the flexible member comprises a helically arranged band forming a spiral like structure around the support member. This is an alternative way of designing the air flow path.

In one aspect the flexible member and the support member are permanently fixed to each other. Thus, the flexible member will be an integrated part of the support member and will thus, not risk remaining inside the nose when the user pulls out the nose plug by means of the support member. In one aspect, the flexible member and the support member are integrally formed in a single piece of material.

In another preferred aspect the nose plug comprises two flexible members or two sets of flexible members connected to each other by means of a generally U-shaped support member, each flexible member being attached at one leg of said U-shaped support member. The extension of the support member between the two flexible members makes the nose plug easy to place within both nostrils at the same time and easy to remove. This extension also hinders the nose plug to be placed to deep into a nostril, which could damage soft tissue.

In one aspect, the anti-microbial substance is selected from the group consisting of anti-viral agents, anti-bacterial agents, and anti-mycotic agents.

In one aspect, the anti-microbial substance comprises silver ions.

In one aspect, the flexible member is made of a thermoplastic elastomer and the anti-microbial substance is evenly dispersed therein.

In one aspect, the flexible member is coated with a coating comprising the anti-microbial substance.

### BRIEF DESCRIPTION OF THE DRAWINGS

The present disclosure will now be described in more detail, and with reference to the accompanying drawings, in which:

FIG. 1 shows an aspect of a nose plug in a perspective view,

FIG. 2 illustrates the nose plug in FIG. 1 from above,

FIG. 3 illustrates a cut along A-A in FIG. 2,

FIG. 4 illustrates a cut along B-B in FIG. 2,

FIG. 5 shows a nose plug moulded by using a 2K technique,

FIG. 6 illustrates an aspect of a nose plug,

FIG. 7 shows a helical aspect of a nose plug in a perspective view, and

FIG. 8 shows a helical aspect of a nose plug from above.

### DETAILED DESCRIPTION

In the following description, the disclosure will be described in more detail with reference to certain aspects and to the accompanying drawings. For purposes of explanation and not limitation, specific details are set forth, such as particular scenarios, techniques, etc., in order to provide a thorough understanding of the present disclosure. However, it is apparent to one skilled in the art that the present disclosure may be practised in other aspects that depart from these specific details.

The expression nose plug used in this application is not a stopper in the sense to close the nostrils completely. While it fills the nostril, thereby hindering air from straight into the nose, it is not intended to hinder air from entering. In fact, the disclosed nose plug may for many users actually enhance ease of flow of air in and out of the nose, as the configuration of the flexible members may widen the nostril slightly and/or support a more open shape of the nostril when placed in the nostril. Through enabling air to flow in a tortuous structure, thus over a longer distance than without the nose plug, inactivation of infectious agents by the anti-microbial substance in a material in contact with the air flow is facilitated.

The anti-microbial substance is preferably permanently immobilized in the material of the flexible members or on the surface thereof. That is, the substance is preferably not released under normal usage and storage conditions. In one aspect, the anti-microbial substance is permanently immobilized within the material of the flexible member, such as dispersed therein. This minimizes the risk of unintended release of the anti-microbial substance from the material. Avoidance of release of the anti-microbial substance from the material may be desirable for certain anti-microbial substances and/or certain categories of users. For instance, anti-microbial substances comprising silver ions are not degraded in the gastrointestinal tract by humans and may thus be excreted to the environment where the anti-microbial effect may cause environmental harm. Anti-microbial substances may also have potential negative side effects if released to any user or to a user of a certain category, such as children, why it may be desirable to avoid release of the anti-microbial substance while still allowing it to exert its anti-microbial effect on any potential infectious agent present in the air flowing through the nose plug. The anti-microbial substance may preferably be a silver ion containing composition.

In one aspect, the anti-microbial substance comprises silver ions encapsulated in glass. In other aspects, the anti-microbial substance comprises silver ions provided in a salt, as a fluid and/or encapsulated in a ceramic or other suitable carrier.

One material suitable for incorporation as a flexible member and optionally as a support member in the nose plug according to the present disclosure may be prepared by mixing 3 parts of a silver ion containing composition (e.g. Sanitized® MB E 99-58, Sanitized AG, Burgdorf, Switzerland) and 97 parts thermoelastic polymer to provide a thermoelastic polymer containing ˜3% anti-microbial substance. Another suitable material may be prepared by mixing 5 parts of a silver ion containing composition (e.g. Sanitized® MB E 99-58, Sanitized AG, Burgdorf, Switzerland) and 95 parts thermoelastic polymer to provide a thermoelastic polymer containing ˜5% anti-microbial substance. The silver ions in such substance compositions are preferably encapsulated in glass. In some aspects, the flexible member(s) may be made of a thermoplastic elastomer and the anti-microbial substance, wherein the anti-microbial substance constitutes a percentage in the range of 2% and 10% of the total material in the flexible member, or more preferably in the range of 3% to 6%. In some aspects, the anti-microbial substance constitutes approximately 3%, 4%, 5%, 6%, 7%, 8%, 9% or 10% of the material in the flexible member.

In one aspect, the anti-microbial substance is releasable from the material of the flexible member. This aspect is particularly relevant if it is desired that the anti-microbial substance is released to a user, such as if the anti-microbial substance is safe and effective in terms of the intended user's health.

In one aspect, the anti-microbial substance is included in a coating composition and coated on at least the flexible member.

The anti-microbial substance dispersed in the flexible material or arranged on the surface of the flexible member may be an anti-viral substance, an anti-bacterial substance, or an anti-mycotic substance, or any other anti-microbial substances. Anti-microbial substances include compositions comprising silver ions. Such compositions are commercially available from a number of suppliers, e.g. Santized AG (Burgdorf, Switzerland) under the trade names Sanitized® BC A 21-41, Sanitized® BC A 21-61, Sanitized® BC A 21-72 and Sanitized® MB E 99-58. Anti-microbial substances also include compositions comprising other metal ions, such as zinc, copper or iron ions.

Further anti-microbial substances having anti-viral activity include amantadine (1-adamantylamine or 1-aminoadamantane), rimantadine, pleconaril, hydrogen peroxide, hypochlorites, silver ions, cupric and ferric ions, per-acids, ethanol, parachlorometaxylenol in a sodium C₁₄-C₁₆ olefin sulfonate, glutaraldehyde, quaternary ammonium salts, chlorhexidine and chlorhexidine gluconate, curdline sulphate, glycerol, lipids, azodicarbonamide, cicloxolone sodium, dichlorisocyanuric acid (sodium salt), benzalkonium salts, disulfate benzamides and benzisothiazolones, congo red, ascorbic acid, nonoxynol-9, para-aminobenzoic acid, bis(monosuccinamide) derivative of p,p′-bis(2-aminoethyl) diphenlyi-C60) (fullerene), merocyanine, benzoporphyrin derivative monoacid ring A, rose bengal, hypericin, hypocrellin A, anthraquinones (such as those extracted from plants), sulfonated anthraquinones and other anthraquinone derivatives, gramicidine, gossypol, garlic (Allium sativum) extract and/or its components, ajoene, diallyl thiosulfinate (allicin), allyl methyl thioulfinate, methyl allyl thiosulfinate, extracts of ledium, extracts of motherworth, extracts of celandine, extracts of black extracts of currant, extracts of coaberry, extracts of bilberry, extract of Cordia salicifolia, steam distillate from Houttuynia cordata (Saururaceae) and/or its components, 5,6,7-trimethoxyflavone (such as obtained from Calicarpa japonica), isoscullarein (5,7,8,4′-tetrahydroxyflavone) (such as obtained from Scutellaria baikalensis) and isoscutellarein-8-methylether, alkaloids and phytosteryl ester compounds.

When an anti-viral substance is incorporated in the flexible member, the nose plug according to the present disclosure may be useful to reduce the viral load of inhaled and/or exhaled air. The nose plug as disclosed may thus be useful in reducing the viral load of inhaled and/or exhaled air, and in methods for preventing, or reducing the risk or severity of, viral infections.

Examples of viruses that can be reduced in the inhaled and/or exhaled air include Enterovirus; Rhinovirus; Adenovirus; Influenzavirus A and B; Norovirus; Coronavirus (e.g. Sars-Cov-1; Sars-Cov-2, Mers-Cov, HCoV-229E); Chickenpox; Rotavirus; Measles; Mumps; Smallpox.

Further anti-microbial substances having anti-bacterial activity include ampicillin and its derivative amoxicillin; quinolones such as lomefloxacin, ofloxacin, norfloxacin, gatifloxacin, ciprofloxacin, moxifloxacin, levofloxacin, gemifloxacin, cinoxacin, nalidixic acid, trovafloxacin, sparfloxacin; aminoglycosides including kanamycin A, amikacin, tobramycin, dibekacin, gentamicin, sisomicin, netilmicin, neomycins B, C and neomycin E (paromomycin); Cephalosporins; Carbepenems; macrolides such as erythromycin, roxithromycin, clarithromycin, azithromycin, and dirithromycin; tetracycline, chlortetracycline, oxytetracycline, demeclocycline, lymecycline, meclocycline, methacycline, minocycline, and tigecycline; chloramphenicol; ticarcillin; rifamycins; penicillin G, benzathine penicillin G, penicillin V, procaine penicillin, propicillin, pheneticillin, azidocillin, clometocillin, and penamecillin; Cloxacillin (dicloxacillin flucloxacillin), methicillin, nafcillin, oxacillin and temocillin; vancomycin, clindamycin, isoniazid, rifampin, ethambutol, pyrazinamide, bacitracin, polymixins, sulfonamides, glycopeptide and nitroimidazoles.

When an anti-bacterial substance is incorporated in the flexible member, the nose plug according to the present disclosure may be useful to reduce the bacterial load of inhaled and/or exhaled air. The nose plug according to the present disclosure may thus be useful in reducing the bacterial load of inhaled and/or exhaled air, and in methods for preventing, or reducing the risk or severity of, bacterial infections.

Examples of bacteria that can be reduced in the inhaled and/or exhaled air include Mycobacterium spp. (incl. Mycobacterium tuberculosis, Mycobacterium bovis); Acinetobacter baumannii; Pseudomonas aeruginosa; Enterobacteriaceae; Enterococcus faecium; Helicobacter pylori; Campylobacter spp.; Salmonella spp.; Neisseria spp.; gonorrhoeae spp.; Streptococcus pneumonia; Haemophilus influenza; Shigella spp.; Staphylococcus aureus (incl. MRSA); Pneumococci; Bacillus anthracis; Neisseria meningitides; Legionella spp.; Cryptococcus spp.; Bordetella pertussis.

Anti-microbial substances having anti-mycotic activity include Amphotericin, nystatin, pimaricin, fluconazole, itraconazole, ketoconazole, naftifine, terbinafine, amorolfine, and 5-Fluorocytosine.

When an anti-mycotic substance is incorporated in the flexible member, the nose plug according to the present disclosure may be useful to reduce the mycotic load of inhaled and/or exhaled air. The nose plug according to the present disclosure may thus be useful in reducing the load of inhaled and/or exhaled air, and in methods for preventing, or reducing the risk or severity of, mycotic infections.

Examples of fungi that can be reduced in the inhaled and/or exhaled air include Aspergillus niger; Blastomyces dermatitidis, Candida spp.

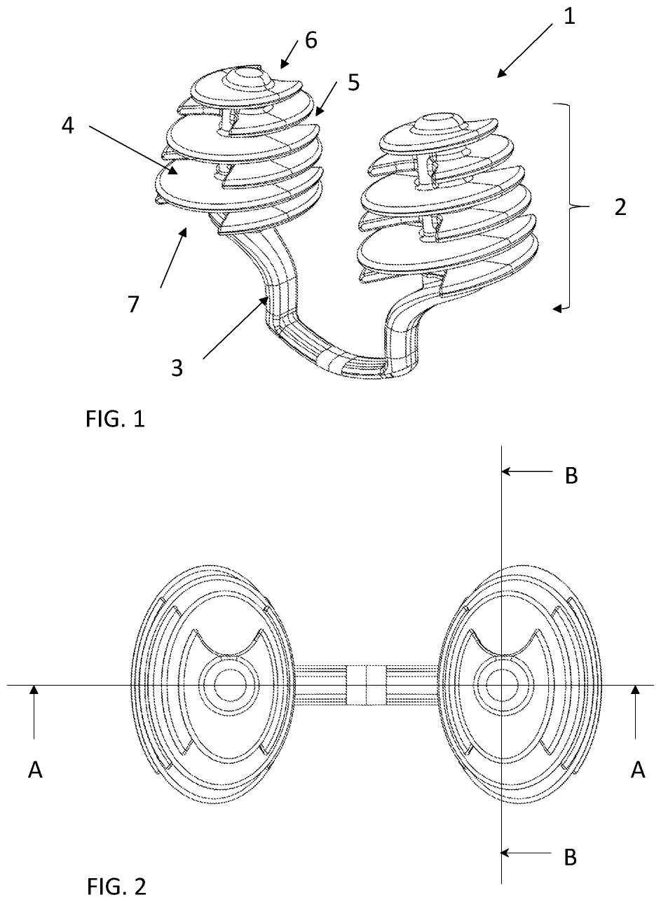

FIG. 1 shows a nose plug 1 comprising a set of flexible members 2 comprising an anti-microbial substance and one rigid, elongated support member 3 adapted to stabilize the flexible member. In this aspect of the nose plug the set of flexible members 2 comprise parallel plates 4 surrounding the support member. Between adjacent plates there are spaces 5. Furthermore, there is provided for communication between theses spaces through the plates. When the nose plug is placed in a nose and the flexible plates accurately fill the openings of the nostrils, air entering the nostrils will, when passing through the nose plug, first enter through a recess 7 in a first lowermost plate. The air flow then passes the nose plug in a tortuous manner and exits through a recess 6 in an uppermost final plate. Exhaled air will flow in the opposite direction. Thus, both when air is inhaled and when air is exhaled, the air will flow over a longer distance than without the nose plug arranged in the nostril, and will be exposed to the anti-microbial substance in both directions.

Throughout the passage through the nose plug, the air is in contact with the flexible member comprising an anti-microbial substance. Any infectious agents comprised in the air will thus be brought into contact with the anti-microbial substance and be inactivated. This aspect illustrates a much extended path for the air to flow through, since every second recess is on a position at the periphery that is opposite on the transverse diameter in relation to the recess on an adjacent plate. The recess 7 of the first plate and the recess 6 of the final plate and all the recesses there between are clearly seen in FIG. 4, i.e. the recess 7 of the first lowermost plate and the recess of every second plate counted from the first plate has the same reference number and the recess 6 of the uppermost final plate and every second plate counted from the final plate has the same reference number. This is valid when the number of plates is an even number (as shown in the aspects in FIGS. 1 to 6), otherwise in the case were the number of the plates is uneven the recess of the first and the final plate will have the same reference number.

In the aspects of a nose plug seen in FIGS. 1 to 6 the set of flexible members 2 comprising an anti-microbial substance is adapted to guide the flow of air entering the nostril in a serpentine or meandering like path. This is achieved when the nose plug is placed in a nostril. The air flow enters the nose plug 1 through the recess 7 of the lowermost plate 4, the air flow is then passing in the space 5 between two adjacent plates, i.e. the first and second plate, arriving at the opposite transverse diametrical end of the first plate, where the air flow is further guided through the recess 6 of the second plate. Thereafter the air flow is passing between two further adjacent plates, i.e. the second and third plate, arriving at the opposite transverse diametrical end of the second plate, where the air flow is further guided through the recess 7 of the third plate. And so on, until the air flow passes between the two uppermost plates and exits into the nostril through the recess 6 of the final plate.

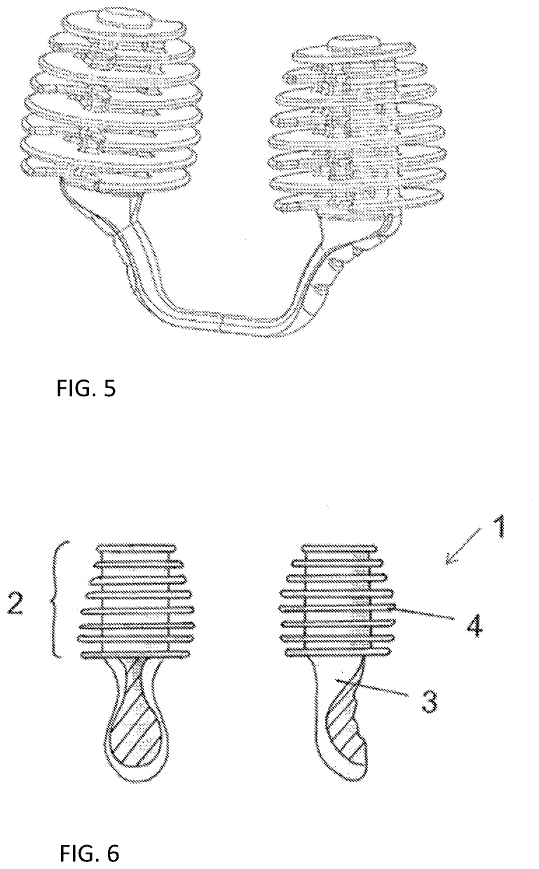

In the aspect illustrated in FIGS. 1 to 4, the number of the plates is 6, and in FIGS. 5 and 6, the number of the plates is 8. The number of the plates may also be another number such as 7, 9 or 10. The number of the plates is preferably ranging from 4 to 12.

FIG. 2 shows the nose plug of the aspect in FIG. 1 from above. As may be seen the plates 4 have an elliptical shape. The final uppermost plate is the smallest (transverse diameter and conjugate diameter) and the plates in the middle, i.e. plate number four and/or five is/are the largest plate(s). The first plate is somewhat larger than the final plate. Seen from the first plate each further adjacent plate has somewhat larger diameters, until the fourth or fifth plate, thereafter each further adjacent plate has somewhat smaller diameters, i.e. from the first to the fourth or fifth plate the transverse and conjugate diameter increases for each plate, from the fourth or fifth plate to the final plate the transverse and conjugate diameter diminishes for each plate. The increase or the diminution of each adjacent plate may be linear, but may also progress irregularly.

In FIG. 2 are the lines A-A and B-B shown. The line A-A coincides with the conjugate diameter of the plates and line B-B coincides with the transverse diameter of the plates.

The flexible members 2 comprise an anti-microbial substance. The anti-microbial substance can be incorporated within the material constituting the flexible member 2, such as evenly dispersed within the material. The anti-microbial substance can also be coated on the flexible member 2, as known in the art.

The flexible member 2 is preferably made of a thermoplastic elastomer, or other suitable polymer, with an anti-microbial substance dispersed evenly throughout the thermoplastic elastomer using any suitable manufacturing technique. In one aspect, the anti-microbial substance may be introduced into the thermoplastic elastomer or other suitable flexible material by using an additive master batch approach. In addition or as an alternative, an anti-microbial substance may be introduced by coating the flexible member and in some aspects, also the support member, with an anti-microbial substance in a suitable solution or dispersed into a thermoplastic elastomer or other suitable material. Thus, the flexible member 2 may be made of a thermoplastic elastomer with an anti-microbial substance coated on the surface.

Other flexible materials may also be used in the flexible member and/or support member. The support member is preferably made of a thermoplastic polymer. Other firm or rigid materials are also possible to use. In some aspects, the support member and/or the flexible member may further comprise one or several reinforcing members, such as an internal core of a more rigid and/or shapeable material, such that parts of the nose plug may be more inflexible and/or the support member may be pressed into a shape to better fit an individual nose or nostril.

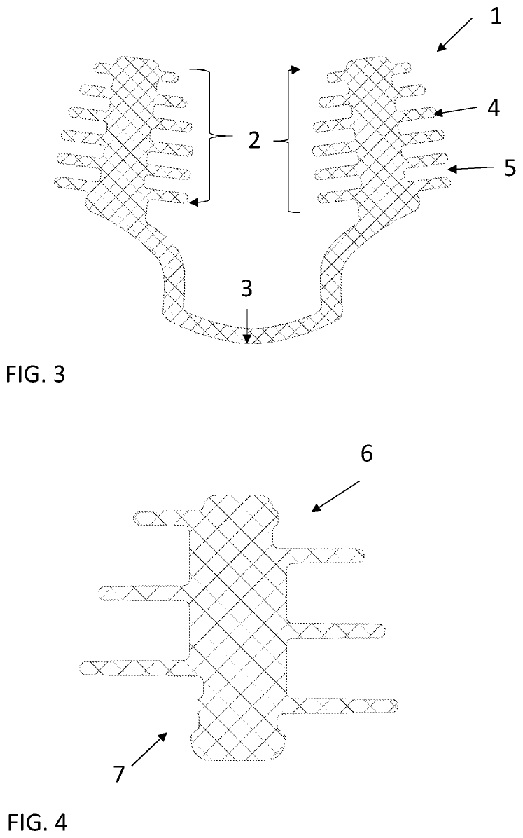

In FIG. 3 a cross-sectional view along A-A be seen. In this figure a set of flexible members 2 are integrally formed with the respective ends of support member 3, and the flexible members 2 surround the ends of support member 3. The support member 3 has a generally U-shaped form and has two sets of flexible members 2 attached thereto, one set on each end. The support member 3 extending between the two ends has a bent shape, in order to be easy to handle.

In FIG. 4 a cross-sectional view along B-B shown. This cut coincides with the transverse diameters of the plates 4. Therefore, the recesses 6 of every second plate are shown on the one side and the recesses 7 of every other second plate are shown on the other side.

As can be seen in FIG. 5, the two legs of the generally U-shaped support member 3 may be slightly inclined and/or angled in relation to each other. This facilitates adapting the nose plug to the extension and arrangement of the nostrils. The plug should be properly arranged within the nose, without widening the nostrils more than necessary.

The nose plug according to the present disclosure may be manufactured by moulding using a 2K technique, where two materials are moulded together. Thus, it is possible to first mould the support member in one material, and thereafter mould the flexible members directly onto the support member, using the same or a different materials. This is illustrated in FIGS. 5 to 7. The method of manufacturing the nose plug may also comprise other techniques, such as extrusion moulding, injection moulding, casting, compression moulding, transfer moulding or any other suitable technique for the material used. In some aspects, only one material may be used for manufacturing the entire nose plug, i.e. the same material for the flexible member as for the support member. The anti-microbial substance will then be dispersed throughout the entire nose plug. Furthermore, the flexible member and the support member comprised in the nose plug according to the present disclosure may also be manufactured by moulding them separately, using the same material or two different materials, and thereafter assembling the two parts together. In such cases, the content of the anti-microbial substance can be adapted to different parts of the nose plug.

The antiviral properties of the material for manufacture of the flexible member and/or the support member of the nose plug may be evaluated according to standard methods, such as ISO 21702. Preferably, properties are evaluated against at least two different types of viruses, such as one enveloped, positive-sense, single-stranded RNA virus (e.g. HCov-229E) and one non-enveloped double-stranded DNA virus (e.g. adenovirus type 5). Examples of such tests and their results are seen in Example 1 below.

The antibacterial properties of the material for manufacture of the flexible member and/or the support member of the nose plug may be evaluated according to standard methods, such as ISO 22196. Preferably, properties are evaluated against at least two different types of bacteria, such as Escherichia coli (ATCC 8739) and Staphylococcus aureus (ATCC 6538P). Examples of such tests and their results are seen in Example 2 below.

Examples of the anti-microbial properties of the nose plug configured as disclosed herein with anti-microbial substance incorporated therein are described in Examples 3 and 4, showing significant reduction of viral and bacterial load, respectively, in air passing through a nose plug with an anti-microbial substance incorporated therein.

The disclosed examples support the conclusion that the combination of the air being led through a longer path, without causing inhibition of air flow, and an incorporated anti-microbial substance in the material of the nose plug and thus along all surfaces of such a path, result in a very effective, comfortable and easy to use nose plug for reducing the risk of being infected and/or infecting other people. More specifically, as mentioned previously, the arrangement of the flexible member of the nose plug allows air to be led through a longer path than in a nostril without a nose plug, while maintaining ease of breathing for a user. When the arrangement of the flexible members is combined with an anti-microbial substance being incorporated in the material, a significant effect of reducing microbial load of inhaled and exhaled air is achieved.

The disclosed examples further point to a long-term effect, concluding that a nose plug as disclosed herein may be worn by a user for at least 8 hours, and still maintain the high effectiveness of reducing microbial load of inhaled and exhaled air. Notably, due to the design of the nose plug, both inhaled and exhaled air are subjected to the same anti-microbial reduction.

FIG. 6 illustrates an aspect, wherein each nose plug, comprises one flexible member arranged on a support member, is adapted to be arranged separately in one nostril each.

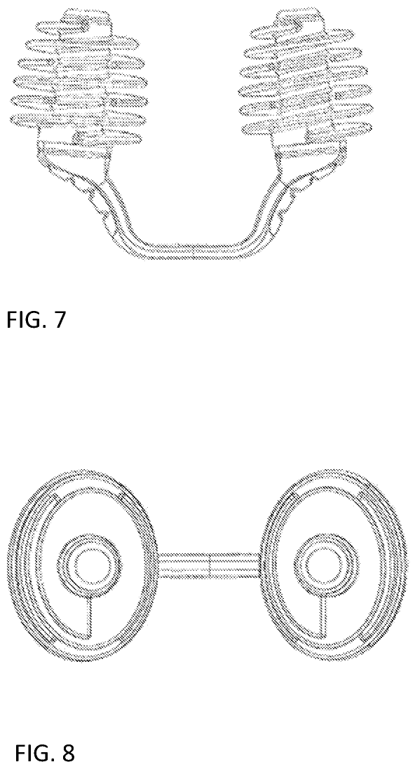

FIG. 7 shows an aspect with a flexible member comprising a helically arranged band in a perspective view. This spiral like structure gives the air entering the nostrils an extended path. The distance of the path may be varied due to the pitch angle of the helical wings of the band. There may also be recesses provided on the helically arranged band. But since air is allowed to enter without recesses, this is not an essential feature.

In FIG. 8, the aspect in FIG. 7 is seen from above. The helically arranged band shown in FIGS. 7 and 8 shows the flexible member being adapted to guide the flow of air entering the nostril in a spiral like path.

Further, the above mentioned and described aspects are only given as examples and should not be limited to the present disclosure. Other solutions, uses, objectives, and functions within the scope of the disclosure as claimed in the accompanying patent claims should be apparent for the person skilled in the art.

### Example 1

The antiviral properties of the material for manufacture of the flexible member and the support member of the nose plug was evaluated according to standard method ISO 21702.

Four different virus strains were inoculated onto the surface of a test material comprising 3 parts of a silver ion containing composition (Sanitized® MB E 99-58, Sanitized AG, Burgdorf, Switzerland) and 97 parts thermoelastic polymer. Antiviral activities of the test material surface using a non-active surface (stainless steel) as control was tested under conditions defined by the ISO 21702 (2019) adapted protocol for a contact time of 8 hours. Under experimental conditions (25° C., 8 hours, 80% RH), the test material surface showed an antiviral activity per cm 2 associated with a logarithmic reduction and viral reduction as summarised in Table 1 below.

TABLE 1

| | Antiviral activity | Viral |
|---|---|---|
| Viral strain | R (log₁₀/cm²) | reduction (%) |
| Murine norovirus (ATCC VR-1937) | R = 1.2 | 93.69 |
| Human rhinovirus A16 (ATCC | R = 1.3 | 94.99 |
| VR283) | | |
| Type 5 adenovirus (ATCC CCL-185) | R = 0.9 | 87.41 |
| Human coronavirus HCov-229E | R = 1.2 | 93.69 |
| (ATCC VR-740) | | |

### Example 2

The antibacterial properties of the material for manufacture of the flexible member and the support member of the nose plug was evaluated according to standard method ISO 22196.

Five different bacterial strains were inoculated onto the surface of a test material comprising 5 parts of a silver ion containing composition (Sanitized® MB E 99-58, Sanitized AG, Burgdorf, Switzerland) and 95 parts thermoelastic polymer. Antibacterial activities of the test material surface was tested under conditions defined by the ISO 22196 (2011) adapted protocol for a contact time of 8 hours. Under experimental conditions (25° C., 8 hours, 80% RH), the test material surface showed an antibacterial activity and antiviral reduction as summarised in Table 2 below.

TABLE 2

| | Antibacterial | Bacterial |
|---|---|---|
| Bacterial strain | activity | reduction (%) |
| Streptococcus pneumoniae (ATCC 6305) | R > 4.10 | 99.99 |
| Klebsiella pneumoniae (ATCC 4352) | R > 5.00 | 99.99 |
| Staphylococcus aureus (ATCC 33592) | R > 4.20 | 99.99 |
| Escherichia coli (ATCC 8739) | R > 5.10 | 99.99 |
| Staphylococcus aureus (ATCC 6538P) | R > 4.80 | 99.99 |

### Example 3

The anti-viral properties of the nose plug configured as disclosed herein with anti-microbial substance incorporated therein is evaluated, showing significant reduction of viral load in air that passes through a nose plug with an anti-microbial substance incorporated therein.

Test articles configured as a nose plug according to the aspect shown in FIGS. 1 to 3 as disclosed herein, but with seven parallel plates in each flexible member, were formed by injection molding using a material comprising 5 parts of a silver ion containing composition (Sanitized® MB E 99-58, Sanitized AG, Burgdorf, Switzerland) and 95 parts thermoelastic polymer.

The test articles were mounted in a simulated nostril, such that air can be applied to the nose plug mimicking inhalation of air in a nostril with a nose plug provided in the nostril.

A test procedure was performed to evaluate the Viral Filtration Efficiency (VFE) of five test articles at an increased challenge level. Notably the term “filtration” herein means removal of infectious agents from an air stream. A suspension of ΦX174 bacteriophage (a single-stranded DNA (ssDNA) virus that infects Escherichia coli) was delivered to the test article at a challenge level of greater than 10⁶ plaque-forming units (PFU) to determine the filtration efficiency. The challenge was aerosolized using a nebulizer and delivered to the test article at a fixed air pressure and flow rate of 10 liters per minute (LPM). The aerosol droplets were generated in a glass aerosol chamber and drawn through the test article into all glass impingers (AGIs) for collection. The challenge was delivered for a one minute interval and sampling through the AGIs was conducted for two minutes to clear the aerosol chamber. The mean particle size (MPS) control was performed at a flow rate of 28.3 LPM using a six-stage, viable particle, Andersen sampler for collection. The titer of the AGI assay fluid was determined using standard plaque assay techniques.

The filtration efficiency percentages were calculated using the following equation:

$$\% VFE = \frac{C - T}{C} \times 100$$

wherein C=Challenge Level and T=Total PFU recovered downstream of the test article. Notably the term “filtration” herein means removal of infectious agents from an air stream. The results are shown in Table 3 below.

TABLE 3

| | Total PFU | Filtration efficiency |
|---|---|---|
| Test article number | recovered | (%) |
| 1 | 1.4 × 10⁶ | 68 |
| 2 | 1.3 × 10⁶ | 71 |
| 3 | 5.0 × 10⁵ | 89 |
| 4 | 1.5 × 10⁶ | 67 |
| 5 | 4.7 × 10⁵ | 90 |

### Example 4

The anti-bacterial properties of the nose plug configured as disclosed herein with anti-microbial substance incorporated therein is evaluated, showing significant reduction of bacterial load in air that passes through a nose plug with an anti-microbial substance incorporated therein.

Test articles configured as a nose plug according to the aspect shown in FIGS. 1 to 3 as disclosed herein, but with seven parallel plates in each flexible member, were formed by injection molding using a material comprising 5 parts of a silver ion containing composition (Sanitized® MB E 99-58, Sanitized AG, Burgdorf, Switzerland) and 95 parts thermoelastic polymer.

The test articles were mounted in a simulated nostril, such that air can be applied to the nose plug mimicking inhalation of air in a nostril with a nose plug provided in the nostril.

A test procedure was performed to evaluate the Bacterial Filtration Efficiency (BFE) of five test articles at an increased challenge level. A suspension of Staphylococcus aureus, ATCC #6538, was delivered to the test article at a challenge level of greater than 10⁵ colony forming units (CFU). The challenge was aerosolized using a nebulizer and delivered to the test article at a fixed air pressure and flow rate of 10 liters per minute (LPM). The aerosol droplets were generated in a glass aerosol chamber and drawn through the test article into all glass impingers (AGIs) for collection. The challenge was delivered for a one minute interval and sampling through the AGIs was conducted for two minutes to clear the aerosol chamber. The mean particle size (MPS) control was performed at a flow rate of 28.3 LPM using a six-stage, viable particle, Andersen sampler for collection. The titer of the AGI assay fluid was determined using standard spread plate and/or membrane filtration techniques.

The filtration efficiency percentages were calculated using the following equation:

$$\% BFE = \frac{C - T}{C} \times 100$$

wherein C=Challenge Level and T=Total PFU recovered downstream of the test article. Notably the term “filtration” herein means removal of infectious agents from an air stream. The results are shown in Table 4 below.

TABLE 4

| | Total PFU | Filtration efficiency |
|---|---|---|
| Test article number | recovered | (%) |
| 1 | 4.8 × 10⁴ | 89 |
| 2 | 5.8 × 10⁴ | 86 |
| 3 | 4.5 × 10³ | 98.9 |
| 4 | 3.2 × 10⁴ | 92.5 |
| 5 | 8.6 × 10³ | 98.0 |

1. A nose plug, comprising:
at least one flexible member; and
one rigid, elongated support member,
wherein the flexible member is arranged to surround the support member,
wherein the flexible member is configured to be placed within a nostril to accurately fill an opening of the nostril, and is provided with means for guiding the flow of air entering the nostril in a serpentine or spiral path by means of a plurality of plates or a band,
wherein the flexible member comprises an anti-microbial substance permanently immobilized in a material of the flexible member or on a surface of the flexible member,
wherein the anti-microbial substance is configured to significantly reduce a number of viable microbes in the flow of air passing through the serpentine or spiral path of the nose plug, and
wherein the anti-microbial substance comprises metal ions selected from the group consisting of silver ions, zinc ions, copper ions, and iron ions.

2. The nose plug according to claim 1, wherein a set of flexible members comprise a plurality of parallel plates arranged along the support member, and
wherein each plate extends in a direction perpendicular to the extension of the support member.

3. The nose plug according to claim 2, wherein the plates have differently sized peripheries, in order to adapt the nose plug to a shape of the inside of a nostril.

4. The nose plug according to claim 2, wherein each plate has one recess at the periphery, and
wherein the recesses are arranged such that no recess overlaps a recess in a previous or subsequent plate.

5. The nose plug according to claim 2, wherein a first set of recesses are arranged on every second plate on a position at the periphery at one end of a transverse diameter, and
wherein a second set of recesses are arranged on every plate therebetween at the periphery at another end of the transverse diameter.

6. The nose plug according to claim 1, wherein the flexible member comprises a helically arranged band forming a spiral structure around the support member.

7. The nose plug according to claim 1, wherein the anti-microbial substance comprises silver ions.

8. The nose plug according to claim 7, wherein the silver ions are encapsulated in glass.

9. The nose plug according to claim 1, wherein the flexible member is made of a thermoplastic elastomer, and the anti-microbial substance is evenly dispersed therein.

10. The nose plug according to claim 1, wherein the flexible member is made of a thermoplastic elastomer and the anti-microbial substance, and
wherein the anti-microbial substance constitutes a percentage in a range of 2% and 10% of the total material in the flexible member.

11. The nose plug according to claim 1, wherein the flexible member is coated with a coating comprising the anti-microbial substance.

12.-14. (canceled)

15. The nose plug according to claim 2, wherein the set of flexible members are made of a thermoplastic elastomer, and the anti-microbial substance is evenly dispersed therein.

16. The nose plug according to claim 2, wherein the set of flexible members are made of a thermoplastic elastomer and the anti-microbial substance, and
wherein the anti-microbial substance constitutes a percentage in a range of 2% and 10% of the total material in the flexible member.

17. The nose plug according to claim 2, wherein the set of flexible members are coated with a coating comprising the anti-microbial substance.
